(19) United States
(12) Patent Application Publication
(10) Pub. No.: US 2022/0387436 A1
(43) Pub. Date: Dec. 8, 2022

(54) BOVINE SUPPLEMENT FOR NEONATAL CALVES

(71) Applicant: TechMix, Inc., Stewart, MN (US)

(72) Inventors: Nathan C. Upah, Clutier, IA (US); Aaron N. Fritchen, Des Moines, IA (US); Dennis M. McKilligan, Ames, IA (US); Bradley W. Kolstad, Waconia, MN (US)

(21) Appl. No.: 17/351,598

(22) Filed: Jun. 18, 2021

Related U.S. Application Data
(63) Continuation-in-part of application No. 16/117,227, filed on Aug. 30, 2018, now Pat. No. 11,298,357.

(51) Int. Cl. A61K 31/522; A61K 47/46; A61K 47/44; A61K 47/12; A61K 47/26; A61K 47/36; A61K 47/02; A61K 9/00; A23K 20/137; A23K 10/30; A23K 20/163; A23K 20/158; A23K 20/105; A23K 20/121; A23K 50/10; A23K 50/60; A23K 40/00

(52) U.S. Cl. CPC A61K 31/522; A61K 47/46; A61K 47/44; A61K 47/12; A61K 47/26; A61K 47/36; A61K 47/02; A61K 9/006; A23K 20/137; A23K 10/30; A23K 20/163; A23K 20/158; A23K 20/105; A23K 20/121; A23K 50/10; A23K 50/60; A23K 40/00

(57) ABSTRACT

The present invention relates generally to a feed supplement preparation for administration to ruminant animals and, more particularly, to a formulation for an oral bovine supplement for neonatal calves that delivers naturally occurring caffeine, in a stable liquid form of elevated viscosity and optionally antioxidants and other electrolytes. A 10-40 ml liquid dose of the supplement containing green tea extract delivers a concentrated amount of caffeine of about 100-400 mg to the neonatal calf.

### CROSS-REFERENCE TO RELATED APPLICATIONS

This application is a continuation-in-part of U.S. Ser. No. 16/117,227 filed on Aug. 30, 2018, which is hereby incorporated by reference in its entirety.

### FIELD OF THE INVENTION

The present invention relates generally to feed supplement preparations for administration to ruminant animals and, more particularly, to a formulation for an oral bovine supplement for neonatal calves that delivers naturally occurring caffeine and a viscosity adjustor in a stable liquid form for absorption through the oral mucosal of the neonatal calf.

### BACKGROUND OF THE INVENTION

“Bovine dystocia” refers to a state during the birthing process where it becomes difficult or impossible for the cow to deliver her calf without human assistance. It can occur during the first or second stage of labor, and usually involves the calf being misaligned backwards or even sideways in the birth canal, or otherwise unsuited for the birth canal's shape and size. The incidence of dystocia in dairy cattle has been reported to range from 2-22%, while the proportion of cows requiring human assistance during labor ranges from 10-50%.

Contributing factors for bovine dystocia include calf birth weight and the size of the pelvic area of the mother cow (“heifer” or “dam”). Thus, dairy and beef cattle operations will often cull heifers with small pelvic areas from their breeding programs and intentionally use bulls that are proven to sire calves with small birth weights. Providing proper nutrition to the heifer prior to the labor process can also ensure her ability to exert greater propulsive forces for passing the calf through the birth canal. However, even with proper herd management and nutrition policies, many incidents of bovine dystocia still occur. Given the cost and time expended in impregnating and gestating heifers and cows, as well as potential injuries or death for the mother or calf due to dystocia, this can pose a substantial economic risk for a dairy or cattle operation.

Indeed, calves requiring human assistance during the labor process experience a 50% greater risk of mortality. During the calving process, the fetus experiences neonatal asphyxia, resulting in low blood oxygen levels and areas of decreased blood flow referred to as “hypoxia.” Such hypoxia can progress to anoxia (no oxygen in the blood). Prolonged anoxia can lead to fetal death within six minutes.

Newborn calves require immediate colostrum supplementation shortly after birth. But, hypoxic neonatal calves often suffer from a state of drowsiness and recumbency that interferes with their physical ability to stand and nurse colostrum. Additionally, the ability of the neonatal calf to absorb the immunoglobulins and other components of colostrum is compromised if the neonatal calf is in a hypoxic state. An unchecked hypoxic state can be prolonged for multiple hours and could potentially overlap with naturally occurring gut closer, thereby creating a secondary scenario to impair or completely halt the ability of passive transfer of immunoglobulins from colostrum. This factor further compounds the need to aid in the rapid oxygenation and vigor of the neonatal bovine. Moreover, lethargic calves will typically receive supplemental quantities of glucose too in an attempt to stimulate muscle contraction and overall vigor. But, such glucose supplementation may produce an insulin response that decreases appetite in the neonatal calf that results in periods of hypoglycemia that further amplifies lethargy. This vicious circle can be extremely detrimental to the neonatal calf during the first 12-24 hours of the neonatal calf's life when appetite is crucial for colostrum intake.

Caffeine has been documented as effective intervention to alleviate apnea episodes for human babies. However, caffeine is not considered to be a required nutrient for livestock, so synthetic caffeine lacks “generally regarded as safe” (“GRAS”) status by the Association of American Feed Control Officials (“AAFCO”). Thus, it would be considered illegal to administer human supplements containing synthetic caffeine to treat hypoxia in calves. But, tea is given GRAS status by AAFCO, and tea leaves contain naturally occurring forms of caffeine.

Various nutritional supplement products containing caffeine are known within the industry for humans for purposes of improving health. For example, U.S. Published Application 2011/0123651 filed by Mower et al. discloses a nutritional beverage used to combat the aging process in humans. It contains resveratrol that can be sourced from grape skins or in the form of wine produced from grapes and grape skins. Also included within the beverage is water, a suspension agent like sodium citrate, and optionally quercetin which is an antioxidant and anti-inflammatory agent. Mower further discloses that the beverage will also include catechins that are polyphenols mostly derived from green tea leaf extract that promote genetic stimulation for increased proliferation of beneficial cells in humans.

However, this Mower anti-aging supplement product only includes caffeine as a secondary ingredient on a trace basis (i.e., 0.067% wt). He also provides his product in liquid form which is “buccally delivered” to the human customer by which the human must retain the beverage in his mouth for one minute before swallowing. Needless to say, a neonatal calf cannot be expected to delay swallowing a liquid supplement product for one minute, while retaining it in its mouth in order to produce delivery of an ingredient like resveratrol or caffeine through the inside of the mouth. Towards this end, Mower only includes a very small amount of xanthan gum as a thickener, since his product is delivered in liquid form free of mucosal adsorption.

U.S. Published Application 2008/0038409 file by Nair et al. discloses a “cocoa water beverage” made from water steeped in a coca product or powder. The resulting product is consumed by a human to stimulate the immune system, improve heart health, detoxify, recuperate muscles, enhance cognitive abilities, etc. However, Nair's corn syrup is used as a sweetener agent, instead of as a viscosity adjustor. Likewise, his vegetable oil ingredient is used to prevent oxidation of the polyphenol and flavanol compounds extracted from the coca products. He does not use his vegetable ingredient to facilitate the suspension of the green tea extract in the neonatal bovine dietary supplement.

Food products are known within the industry that contain caffeine, including caffeine that is sourced from green tea extract. For instance, U.S. Pat. No. 5,879,733 issued to Ekanayake et al. illustrates green tea extract beverages, wherein the green tea extract is acid extracted, run through a cation exchange resin to remove metal cations that produce turbidity, and then run through nano filtration to remove higher molecular weight components that harm the color and clarity of the beverage. But, not only is Ekanayake's product in liquid form that is unsuitable for absorption through the oral mucosal lining of the neonatal calf, but also its 0.5% wt of the green tea concentrate found in the sugar-sweetened beverage is significantly lower than the level required to stimulate a neonatal calf suffering from dystocia or hypoxia. Indeed, the Ekanayake reference discloses a 992 ppm caffeine level in his final product. At 1000 ppm being equivalent to 0.1% wt, 992 ppm caffeine translates to 0.0992% wt caffeine, which is very low.

Meanwhile, U.S. Pat. No. 5,624,698 issued to Dake et al. teaches syrup products used in a “soda fountain” to dispense dilute juice beverages. The syrup comprises a flavor component like green tea or black tea together with the juice flavor. An oil component like a vegetable oil provides desired opacity and cloud to the resulting juice beverage. In order to prevent dispersion of the oil component that would cause it to separate out from the syrup, xanthan gum is added to the syrup as an emulsifying agent.

Even if one were to feed Dake's syrup product to a neonatal calf, it would fail to treat the effects of dystocia. The viscosity of the product at less than 250 cps, preferably less than 150 cps, is simply too low for mucosal delivery of the product to the neonatal calf. By contrast, Dake means his syrup product to be added to a fountain beverage to be drunk by a human. Moreover, Dake only uses his black or green tea ingredient (<0.5% wt) as a flavorant, instead of as a stimulating source of caffeine. Furthermore, Dake makes no mention of a target caffeine content for the tea in his final solution.

Even the popular “energy drinks” that are consumed by humans to provide rapid stimulation are unsuitable for treating neonatal calves suffering from dystocia. For example, U.S. Published Application 2017/0231996 filed by Levi et al. discloses energy drinks containing supersaturated caffeine levels. They contain 1.7-3.0% (w/v) levels of caffeine. They are also disclosed as containing 150-250 mg caffeine in 9 ml water. However, this 150 mg in 9 ml water dose only delivers 1.66% wt of caffeine. This is a surprisingly low caffeine level. Moreover, Levi seems to use synthetic caffeine in the form of caffeine citrate, instead of the naturally occurring caffeine source required by AAFCO regulations for food animals.

Even Clif Bar & Co.'s popular “Clif Shot Turbo Double Espresso Energy Gel” product and the even more popular “5-Hour Energy” drink only contain 0.24% wt and 0.43% wt caffeine levels, respectively. Furthermore, the 5-Hour Energy product has a viscosity level <150 cps, which is significantly lower than the viscosity level required for mucosal delivery to a neonatal calf. Hence, despite what the “energy gel” term suggests, these types of products are not very viscous, and certainly would be incapable of retaining in the neonatal calf's mouth and delivering caffeine via the mucosal lining.

Thus, it would be highly beneficial to provide a bovine supplement for neonatal calves that contains non-synthetic caffeine as a stimulant for prompting the calves to consume colostrum and stimulate their respiration and reduce edema to enable them to survive the effects of bovine dystocia and hypoxia. Such a bovine supplement should exhibit a relatively high viscosity level for mucosal delivery of the supplement's ingredients to the calves.

### SUMMARY OF THE INVENTION

A neonatal bovine dietary supplement that is provided to a newborn calf to enhance nutritional intake during this vulnerable early stage of the calf's life is provided by the invention. Bovine dystocia can cause systemic depression and recumbency that weakens the neonatal calf and prevents it from standing up to consume colostrum. The neonatal bovine dietary supplement comprises non-synthetic caffeine obtained from a natural source like green tea extract and viscosity adjustors like corn syrup and xanthan gum or guar gum that increase the viscosity of the liquid supplement to about 400-800 centipoise at 21° C., so that the supplement is absorbed by the neonatal calf through its oral mucosal lining in its mouth and tongue. The specific gravity (relative density) of the supplement product is preferably in a range of about 0.9-1.6. The neonatal bovine dietary supplement may optionally include antioxidants and electrolytes.

In this manner, the supplement reliably provides the caffeine to the neonatal calf to increase its energy level, stimulate respiration, and reduce edema that otherwise may thwart the neonatal calf's crucial nutritional intake of colostrum and other animal feed ingredients within the hours following birth. Moreover, the concentrated caffeine level in the green tea extract allows a 10-40 ml dose of the liquid solution supplement product to contain about 100-400 mg of caffeine for delivery to the neonatal calf without filling its stomach which might otherwise prompt it to stop consuming colostrum and other critical animal feed ingredients. Moreover, this novel formulation for a bovine supplement for neonatal calves delivers naturally occurring caffeine in a stable liquid form at elevated viscosity that may optionally include antioxidants, and other electrolytes. The supplement successfully incorporates natural caffeine in a highly concentrated liquid oral dose formulation in which the viscosity of the liquid supplement is such that it facilitates adherence to the tongue and oral cavity, thereby facilitating mucosal absorption, which greatly enhances the effectiveness of the supplement. Additionally, due to the concentrated nature of the product, caffeine can be delivered with minimal volume, thereby minimizing satiety, resulting is ample capacity to intake sufficient quantities of colostrum.

The formula successfully adds naturally occurring caffeine in the form of green tea, which may be an extract, which is also rich in antioxidants and contains a high concentration of caffeine in a unique mix with xanthan gum or guar gum, corn syrup, water and electrolytes. The corn syrup and xanthan gum are added to adjust the viscosity so that the solution sticks to the oral cavity and tongue to allow more rapid absorption of caffeine.

### DETAILED DESCRIPTION OF THE PREFERRED EMBODIMENTS

A unique formulation for a neonatal bovine calf supplement to be administered following birth that delivers naturally occurring, concentrated dose of caffeine sourced from, e.g., green tea extract, antioxidants, and electrolytes for overcoming the adverse effects of dystocia is provided by the invention. The supplement successfully incorporates a viscosity adjustor like xanthan gum or guar gum and corn syrup to produce a product which remains shelf stable in a liquid form, while greatly enhancing the effectiveness of the supplement for absorption through the oral mucosa of the neonatal calf for rapid treatment. The supplement not only provides needed multi-system (i.e., CNS/cardiac/respiratory) stimulation to the newly borne calf, but it also stimulates respiration, reduces edema, and stimulates muscle contraction. In this manner, the supplement treats the three biggest problems encountered by hypoxic calves: lethargy, respiratory rate, and high concentrations of adenosine.

The neonatal bovine dietary supplement of the present invention comprises a concentrated dose of non-synthetic caffeine and water in a liquid solution that is modified by a viscosity adjustor to produce a viscosity of about 400-800 centipoise (“cps”) at 21° C., preferably about 500-650 cps, even more preferably about 600 cps, so that when the supplement is introduced into the mouth of the newly-born calf who is suffering from dystocia or hypoxia, the supplement sticks to the neonatal calf's tongue and internal cheeks in the mouth for rapid absorption of the caffeine through the mucosal lining. Due to the close proximity of blood vessels to the oral mucosal surface of the neonatal calf, this allows for much more rapid entry of the caffeine into the circulation system compared with the digestive system that could take multiple hours for an ingested product to enter the circulatory system. The neonatal bovine dietary supplement may contain a number of other optional ingredients, including antioxidants, electrolytes, solubility aides, anti-foaming agents, and preservatives for producing a stable solution that enhances the health of the neonatal calf who is under duress.

For purposes of the neonatal bovine dietary supplement, the non-synthetic caffeine may be sourced from a variety of naturally occurring caffeines that have not been chemically synthesized. Such non-synthetic caffeine sources include green tea, green tea extract, black tea, coffee, and cocoa. Green tea extract having a caffeine level of about 90%, preferably about 99%, is preferred. The non-synthetic caffeine ingredient should comprise about 1-15% wt, preferably about 1-5% wt, of the formulated neonatal bovine dietary supplement product.

The water component of the neonatal bovine dietary supplement product may constitute tap water or deionized water. The water should be clean and free of any contaminants that would impair the health of the neonatal calf. The water ingredient should comprise about 30-60% wt, preferably 35-50% wt, even more preferably about 39% wt, of the formulated neonatal bovine dietary supplement product.

One or more thickening agents should be added to the solution containing water, non-synthetic caffeine, and tri-sodium citrate dihydrate to obtain the desired viscosity level of about 400-800 cps at 21° C., preferably about 500-650 cps, even more preferably about 600 cps. Corn syrup may be used to enhance the viscosity of the resulting neonatal bovine dietary supplement product, although other suitable substitute viscosity-enhancing ingredients include molasses, glycerin, vegetable oil, glucose syrup, and high fructose syrup. This corn syrup or substitute viscosity enhancer should comprise about 40-75% wt, preferably about 50-60% wt, of the formulated neonatal bovine dietary supplement product.

A second viscosity adjustment component for further thickening the neonatal bovine dietary supplement product can be xanthan gum, although guar gum, corn starch, potato starch, or an acid like citric acid, formic acid, ascorbic acid, acetic acid, or phosphoric acid may be used. This further thickening agent should only comprise about 0.05-5.0% wt, preferably about 0.05-0.20% wt, of the formulated neonatal bovine dietary supplement product.

The neonatal bovine dietary supplement of the present invention may also contain one or more antioxidant agents. Such antioxidant agents will address the buildup of free radicals in the neonatal calf caused by a lack of oxygen during the labor process. Caffeine functions as an antioxidant for purposes of the supplement product of the present invention. As noted above, suitable non-synthetic sources of caffeine include green tea, green tea extract, black tea, coffee, and cocoa. It is convenient, therefore, to have the caffeine source perform double duty within the product formulation by additionally providing the antioxidant function. Green tea extract is preferred for contributing antioxidants to the neonatal calf.

Sodium citrate can be added to the product formulation to help to solubilize the caffeine component into solution to make the antioxidants more accessible to the neonatal calf. Sodium citrate will also reduce metabolic acidosis in the neonatal calf caused by hypoxia, or water imbalance. It buffers the blood in the neonatal calf to a proper pH level.

The resulting sodium dissociated from the sodium citrate also serves as an electrolyte for the neonatal calf. Such an electrolyte will help to manage the hydration status of the newborn calf. Sodium helps with absorption and storage of water within the animal Other suitable electrolytes for purposes of the neonatal bovine dietary supplement product formulation include sodium chloride and potassium chloride.

Tri-sodium citrate dihydrate is the preferred form of sodium citrate for purposes of the formulation. Such sodium citrate should comprise about 1-10% wt, preferably about 1-5% wt, of the formulated neonatal bovine dietary supplement product.

Trans-400 represents a 100% active, food-grade, defoaming agent designed to destroy foam in aqueous environments that builds up during the food product manufacturing process. It may be obtained from Applied Material Solutions, Inc. of Elkhorn, Wisconsin. As a vegetable oil-based defoamer, it may be added to the neonatal bovine dietary supplement product in the United States and some foreign countries in order to release bubbles that accumulate during the manufacturing process, so that the bubbles escape to produce a smooth, even end product. If used, Trans-400 should comprise about 0.0-1.0% wt, preferably about 0.1-0.5% wt, of the formulated neonatal bovine dietary supplement product.

Cassia oil is a flavor enhancer that also acts as a preservative and antimicrobial agent. Cinnamaldehyde is its essential component. It may be added to the neonatal bovine dietary supplement product at an about 0.1% wt level. Any phenol propionoid such as oregano oil may be used as a substitute in the product formulation.

Polysorbate 80 liquid is an emulsifier that helps oil components within the neonatal bovine dietary supplement product to integrate into water. Thus, it represents a solubility aid. It is derived from polyethoxylated sorbitan and oleic acid, and it is widely available from a number of manufacturing sources. It should comprise about 0.0-3.0% wt, preferably about 0.07-0.70% wt, of the formulated neonatal bovine dietary supplement product.

Citric acid may be added to the neonatal bovine dietary supplement product as a preservation agent. It should comprise about 0.5-3.0% wt, preferably about 1.2-1.6% wt, of the formulated neonatal bovine dietary supplement product.

A coloring agent may likewise be added to the neonatal bovine dietary supplement product. The formulated product is naturally milky white in color. FD&C Blue Dye or beta-carotene are suitable coloring agents.

One successful formula for the neonatal bovine dietary supplement of the present invention had the following preferred range of ingredients.

| | MIN (% | MAX (% |
|---|---|---|
| INGREDIENT | wt) | wt) |
| Water | 35.00% | 50.00% |
| Green Tea Extract | 1.00% | 5.00% |
| Trisodium Citrate Dihydrate | 1.00% | 5.00% |
| Polysorbate 80 Liquid | 0.0% | 0.7% |
| Trans 400 - Vegetable oil defoaming agent | 0.00% | 0.005% |
| Cassia Oil | 0.0% | 1.0% |
| Corn Syrup Blend | 50.00% | 60.00% |
| Citric Acid | 1.2% | 1.6% |
| Xanthan Gum or Guar Gum | 0.05% | 0.20% |
| FD&C Blue Dye | ~.005% | ~.005% |

The supplement is a homogeneous solution when heated and packaged that becomes a suspension at room temperature. The solution is preferably bottled when warm. The supplement is shelf-stable for a time period greater than two hours, and does not require a factory vacuum seal and it does not grow mold, yeast or bacteria.

The process for preparing the neonatal bovine dietary supplement product of the present invention is as follows:

1. Add the water to a jacketed tank mixer vessel having an impeller blade. Heat the water with a lid to about >50° C., preferably about 50-75° C., even more preferably about 52-57° C.

2. Add half of the xanthan gum or guar gum thickening agent to the vessel, mixing the composition at about 250-500 rpm, depending upon the volume of the batch, for about 15 minutes.

3. Add the other half of the xanthan gum or guar gum thickening agent to the vessel and repeat the mixing process. It takes time for the xanthan gum or guar gum to fully hydrate in the water. If all of the xanthan gum or guar gum ingredient were added to the mixture in the vessel at the same time, the composition would thicken too quickly.

At the same time, incorporation of air from the environment should be avoided during the mixing process.

4. Add the non-synthetic caffeine ingredient to the mixer vessel.

5. Add the tri-sodium citrate dihydrate ingredient to the vessel.

6. Turn off the heat to the mixer vessel.

7. Add the cassia oil to the vessel.

8. Add the Polysorbate 80 to the vessel.

9. Add the corn syrup at about 70° F. to the vessel.

10. Add the citric acid to the vessel.

The final mixture for the neonatal bovine dietary supplement product will generally exhibit a viscosity level of about 75-150 cps at the prevailing 50° C. temperature immediately after mixing. It takes several hours for the guar gum or xanthan gum to fully hydrate. Once the caffeine is fully solubilized, caffeine crystals will start to form within 12-24 hours during cooling to room temperature with a subsequent increase in viscosity. Thus, approximately 24 hours after the mixing process is completed, the neonatal bovine dietary supplement product will achieve its desirable viscosity level of about 550-600 cps at 21° C.

The finished product is preferably packaged in 15-20 ml squeeze tubes or syringe plunger. Such squeeze tubes or syringe plunger will conveniently introduce the product into the newborn calf's mouth.

A typical product dose for the neonatal bovine dietary supplement is about 10-40 ml liquid solution containing a concentrated caffeine dose of about 100-400 mg.

The above specification provides a complete description of the components and preparation process for the neonatal bovine dietary supplement product of the present invention. Since many embodiments of the invention can be made without departing from the spirit and scope of the invention, the invention resides in the claims herein appended.

What is claimed is:

1. A neonatal bovine dietary supplement that delivers a dose of a naturally occurring source of caffeine to a neonatal calf, the supplement comprising non-synthetic caffeine and a viscosity adjustor in liquid form that is formulated to produce a viscosity of about 400-800 centipoise at 21° C. such that the supplement is absorbed through the oral mucosal lining of the neonatal calf.

2. The neonatal bovine dietary supplement of claim 1, wherein the viscosity range of the supplement product is about 500-650 cps at 21° C.

3. The neonatal bovine dietary supplement of claim 1, wherein the naturally occurring caffeine is provided by a green tea, green tea extract, black tea, coffee, or cocoa.

4. The neonatal bovine dietary supplement of claim 1, wherein the naturally occurring caffeine comprises about 1-3% wt of the supplement product.

5. The neonatal bovine dietary supplement of claim 1, wherein the viscosity adjustor comprises a thickening agent like corn syrup, molasses, glycerin, vegetable oil, glucose syrup, or high fructose syrup.

6. The neonatal bovine dietary supplement of claim 5, wherein the viscosity adjustor comprises about 50-60% wt of the supplement product.

7. The neonatal bovine dietary supplement of claim 1, wherein the viscosity adjustor comprises a further thickening agent like xanthan gum, guar gum, corn starch, potato starch, citric acid, formic acid, ascorbic acid, acetic acid or phosphoric acid.

8. The neonatal bovine dietary supplement of claim 7, wherein the further thickening agent comprises about 0.05-0.20% wt of the supplement product.

9. The neonatal bovine dietary supplement of claim 1 further comprising an antioxidant.

10. The neonatal bovine dietary supplement of claim 9, wherein the antioxidant comprises green tea extract.

11. The neonatal bovine dietary supplement of claim 1 further comprising vegetable oil defoaming agent for facilitating the suspension of the non-synthetic caffeine in the supplement product.

12. The neonatal bovine dietary supplement of claim 1 further comprising a preservative or antimicrobial agent like cassia oil or citric acid.

13. The neonatal bovine dietary supplement of claim 1 further comprising an emulsifier like Polysorbate 80.

14. The neonatal bovine dietary supplement of claim 1 further comprising electrolytes like sodium citrate, trisodium citrate dihydrate, sodium chloride, or potassium chloride.

15. The neonatal bovine dietary supplement of claim 1, wherein the green tea extract contains a concentrated amount of caffeine so that a 10-40 ml liquid dose of the supplement delivers 100-400 mg caffeine to the neonatal calf.

16. A neonatal bovine dietary supplement that delivers a dose of a naturally occurring source of caffeine to a neonatal calf, the supplement comprising green tea extract, xanthan gum or guar gum, corn syrup, and water, wherein the viscosity of the supplement is about 400-800 centipoise at 21° C. such that the supplement is absorbed through the oral mucosal lining of the neonatal calf.

17. A neonatal bovine dietary supplement that delivers a dose of a naturally occurring source of caffeine to a neonatal calf, the supplement comprising green tea extract, xanthan gum or guar gum, corn syrup, and water, wherein the level of green tea extract in the supplement is about 1-3% wt.

18. A method of use of a neonatal bovine dietary supplement containing a naturally occurring source of caffeine for a new-born calf, said method comprising:
(a) being presented with a newborn calf suffering from bovine dystocia;
(b) administering to the newborn calf a dose of the supplement comprising non-synthetic caffeine and a viscosity adjustor in liquid form that is formulated to produce a viscosity of about 400-800 centipoise at 21° C.; and
(c) wherein the supplement is absorbed through the oral mucosal lining of the newborn calf.

19. The method of claim 18, wherein the naturally occurring caffeine is provided by a green tea extract.

20. The method of claim 18, wherein the viscosity adjustor comprises corn syrup and xanthan gum or guar gum.

21. The method of claim 18, wherein the supplement dose comprises 10-40 ml liquid with the green tea extract containing a concentrated amount of caffeine so that the dose of the supplement delivers 100-400 mg caffeine to the neonatal calf.

22. A method for preparing a neonatal bovine dietary supplement that delivers a dose of a naturally occurring source of caffeine through the oral mucosal lining of a neonatal calf, the method comprising:
(a) adding water to a mixer vessel and heating it to at least 50° C.;
(b) adding a thickening agent like xanthan gum, guar gum, corn starch, potato starch, citric acid, formic acid, ascorbic acid, acetic acid, or phosphoric acid to the mixer vessel;
(c) adding a non-synthetic caffeine like green tea extract, green tea, black tea, or coffee to the mixer vessel;
(d) adding a further thickening agent like corn syrup, molasses, glycerin, vegetable oil, glucose syrup, or high fructose syrup to the mixer vessel;
(e) allowing the admixture to rest for about 24 hours at which point the resulting neonatal bovine dietary supplement exhibits a viscosity of about 400-800 centipoise at 21° C.

23. The method for preparing a neonatal bovine dietary supplement of claim 22 further comprising one or more of the following steps, prior to the admixture resting step (e), of:
(f) adding an antioxidant like caffeine to the mixer vessel;
(g) adding a flavor enhancer like cassia oil to the mixer vessel;
(h) adding an emulsifier like Polysorbate 80 to the mixer vessel;
(i) adding a preservative agent like citric acid to the mixer vessel.
